(19) United States
(12) Patent Application Publication
(10) Pub. No.: US 2024/0366801 A1
(43) Pub. Date: Nov. 7, 2024

(54) HYPERPOLARISATION METHOD AND PRODUCT

(71) Applicant: CAMBRIDGE ENTERPRISE LIMITED, Cambridge, Cambridgeshire (GB)

(72) Inventors: Arnaud COMMENT, Cambridge (GB); Adam GAUNT, Cambridge (GB); Jennifer Sunari LEWIS, Cambridge (GB); Irene MARCO-RIUS, Cambridge (GB); Tian CHENG, Cambridge (GB)

(21) Appl. No.: 18/683,962

(22) PCT Filed: Aug. 16, 2022
(86) PCT No.: PCT/GB2022/052131

§ 371 Date: Feb. 15, 2024

(30) Foreign Application Priority Data
Aug. 16, 2021 (GB) 2111734.6

(51) Int. Cl. A61K 49/10; A61K 49/18

(52) U.S. Cl. CPC A61K 49/10; A61K 49/18

(57) ABSTRACT

In a method for preparing a hyperpolarised sample, for example for a magnetic resonance procedure, a starting solution comprising alpha-ketoglutaric acid and 13C-labelled molecules is frozen to form a frozen solution. The solution is irradiated with ultraviolet and/or visible radiation, to generate free radicals. The frozen solution is then hyperpolarised by applying a magnetic field to the solution while irradiating it with frequency-modulated microwave radiation.

The invention relates to a method for preparing a hyperpolarised sample, or solution, for example for use in magnetic resonance techniques, and to a sample or solution prepared by the method.

The project leading to this application has received funding from the European Research Council (ERC) under the European Union's Horizon 2020 research and innovation programme (grant agreement No 682574).

### BACKGROUND

Hyperpolarisation of a molecule, termed a molecule of interest, dramatically increases the nuclear magnetic resonance (NMR) signal when samples or solutions containing the molecule are used in magnetic resonance (MR) techniques. Hyperpolarisation preferentially orients the nuclear spins of the molecule of interest prior to its introduction or injection into (depending on the MR technique being used) for example a tube, a bioreactor, an animal, or a human being (U.S. Pat. No. 6,466,814). Nuclear spins of molecules in solutions can be hyperpolarised by Dynamic Nuclear Polarisation (DNP).

One prior-art approach to hyperpolarisation is DNP with rapid dissolution. A concentration of about 10-100 mM of free radicals is introduced into a solution containing the molecules of interest. This starting solution is then frozen and introduced into a polariser, which comprises a cryostat operating at a temperature below 4.2 K and a 3.35-10.1 T magnetic field. DNP is most efficient at low temperature (around 1 K) and high magnetic field (3.35-10.1 T). Microwaves with a frequency close to the electron spin resonance (ESR) of the free radicals are delivered to the frozen solution while inside the low-temperature and high-magnetic field environment.

Once the nuclear polarisation has been enhanced by DNP, the frozen solution cannot be simply extracted from the polariser for storage and/or transport because the longitudinal relaxation time (T₁) of the nuclear spins in the frozen state at low field (outside the polariser) is very short in the presence of the 10-100 mM free radicals. Therefore, to retain the enhanced nuclear polarisation long enough to use the hyperpolarised sample in a MR technique, the frozen solution is rapidly dissolved within the high magnetic field of the polariser with a hot solvent, typically water, to reduce the concentration of free radicals (U.S. Pat. No. 7,372,274). For 13C-labelled (carbon 13 labelled) molecules of interest, the T₁ of the 13C nuclear spins in solution, after the frozen solution has been diluted to less than 1 mM free radicals by the solvent, is long enough (typically 1 min) to be used for NMR, MR spectroscopy (MRS), or MR imaging (MRI) experiments.

Before injection into humans, for example in a hospital, the free radicals must be filtered out of the diluted solution and their residual concentration measured in a quality check (QC) procedure to be below an acceptable level. These procedures add delays and additional potential failure points to the process.

In this process, therefore, problems are caused by the need to filter out the stable free-radical compounds after hyperpolarisation and before the solution is used, and the short time available to complete these steps within the 1 minute T₁ of the 13C nuclear spins. To try to address this problem, an alternative process has been proposed (but not yet used in MR techniques on humans) in which the frozen solution contains a photo-reactive species which generates free radicals upon photo-irradiation with light in the ultraviolet or visible (UV-Vis) spectrum at cryogenic temperature, typically below 200 K. DNP is then carried out in a polariser. After DNP the solution is warmed to a temperature above the quenching temperature of the free radicals, typically around 200 K.

This thermalisation process is therefore intended to remove the need for dissolution inside the polariser and to provide an opportunity to extract the hyperpolarised frozen solution from the polariser in its solid state without losing its enhanced nuclear polarisation.

Replacing stable free radicals by non-persistent photo-induced free radicals potentially circumvents the need for filtration before the sample is injected into a human or animal, but only if the photo-sensitive molecule and its breakdown products are biocompatible and can be safely injected at the relevant doses for MR scans (U.S. Pat. No. 10,520,561).

Photo-irradiated pyruvic acid has been used for MR scans in rodents and could possibly be used for MR scans in humans. In this case pyruvic acid is both the molecule of interest and the photo-active species which generates non-persistent free radicals. There is therefore no need to add additional photo-sensitive molecules. This would also be the case for any other molecule of interest that can act as a free-radical precursor under UV-Vis irradiation. However, if the molecule of interest is not a free-radical precursor under UV-Vis irradiation, then a different free-radical precursor molecule must be added. A wide range of photo-sensitive molecules and in particular keto-acids have been proposed as free-radical precursors (U.S. Pat. No. 10,114,088). However, the keto-acids that have been used for DNP to date are not efficiently converted to free radicals and require a disadvantageously high starting concentration to allow the preparation of solutions containing a sufficiently high concentration of hyperpolarised molecules of interest. The high concentration of the free-radical precursor molecules disadvantageously makes the final solution unsuitable for use. For example, to obtain an adequate concentration of free radicals to enable hyperpolarisation, the prior art indicates that at least 1M of free-radical precursor is required in the starting solution (see e.g. I. Marco-Rius et al., J. Am. Chem. Soc. 140, 14455 (2018), A Capozzi et al., Angewandte Chemie 58, 1334 (2019)). Such concentrations raise safety concerns for injection into humans, especially if the precursors are synthetic or exogenous molecules.

### SUMMARY OF INVENTION

The invention provides a method for preparing a hyperpolarised sample, and a hyperpolarised sample, as defined in the appended independent claims to which reference should now be made. Preferred or advantageous features of the invention are set out in dependent subclaims.

The invention may thus advantageously provide a method for preparing a hyperpolarised sample, or for hyperpolarising a molecule of interest, comprising the steps of freezing a solution comprising alpha-ketoglutaric acid (alpha-KG) and 13C-labelled (carbon 13 labelled) molecules (the molecule of interest) to form a frozen solution, irradiating the frozen solution with ultraviolet and/or visible radiation, and hyperpolarising the frozen solution by applying a magnetic field to the solution while irradiating the frozen solution with frequency-modulated microwave radiation.

The inventors have found that this combination of steps provides a particularly, and surprisingly, effective method for hyperpolarising 13C-labelled molecules, and in particular for producing an improved biocompatible hyperpolarised sample which is much more easily usable in MR techniques than conventional samples.

The method uses alpha-KG in the starting solution containing 13C-labeled molecules, followed by photo-irradiation to generate free radicals from the alpha-KG. Low-temperature Dynamic Nuclear Polarisation (DNP) using frequency-modulated microwaves then polarises the 13C-labelled molecules.

The inventors have found that this combination of steps is surprisingly beneficial. In a preferred embodiment, the invention may enable the preparation of a biocompatible hyperpolarised sample which contains an advantageously low concentration of free-radical precursors and recombination products from the thermalisation of the free radicals, in which these products are biocompatible, and which can be stored for up to 48 hours while retaining its hyperpolarisation. This is an enormous improvement on the sample lifetime of about 1 minute which is available for MR techniques in hospitals today.

This improvement relies on a particular combination of features. First, the inventors have found that the free-radical yield (or conversion rate) of the alpha-KG when the solution is exposed to UV-Vis irradiation is unexpectedly very high. The inventors' experiments have achieved yields on the order of 10-50%. By comparison, in prior-art methods using other free-radical precursors, yields of 1-7% are typically achieved. As a result, in embodiments of the invention only a surprisingly low concentration of alpha-KG needs to be admixed into the starting solution containing the molecules of interest before DNP. This is highly advantageous for a sample that will ultimately be used for a magnetic resonance procedure, for example being injected into a human.

While this high free-radical yield is beneficial, it is not sufficient by itself to achieve the remarkable benefits of the invention. The inventors have also found that polarisation of a solution containing free radicals generated from alpha-KG is greatly enhanced by application of frequency-modulated microwaves. This is surprising because free radicals generated by photo-irradiation of alpha-KG exhibit a very narrow electron spin resonance (line width of 6.5 mT at 0.335T). The skilled person, based on their conventional understanding, would not expect modulation of the microwave frequency to be beneficial for DNP using free radicals which exhibit a narrow electron spin resonance. This approach is understood only to be beneficial for free radicals which exhibit a wide electron spin resonance.

Therefore, the skilled person would not consider combining photo-irradiation of alpha-KG with exposure to frequency-modulated microwaves during DNP.

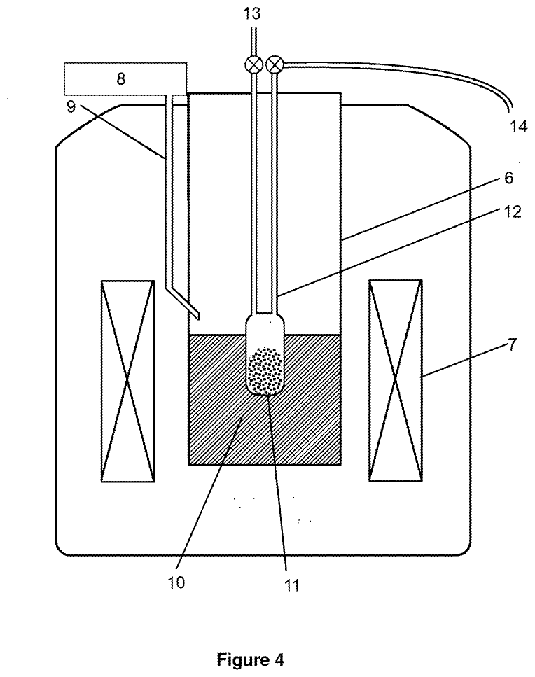

Surprisingly, however, the inventors have found that frequency modulation of the microwaves following photo-irradiation of alpha-KG can enhance the 13C polarisation in methods embodying the invention by a very significant factor, of up to 4 times (as illustrated in FIG. 4 below). The reasons for this effect are not fully understood, but the inventors' observation is leading to research to try to understand this unexpected effect.

In methods embodying the invention, following DNP, a subsequent rapid increase in temperature above 200K may then be applied to force the quenching of the free radicals and yield a radical-free sample with extended 13C T₁.

Preferably, the microwave frequency is modulated at a rate between 1 Hz and 1 MHZ, particularly preferably at a rate above 0.1 KHz and less than 10 kHz, and an amplitude between 1 Hz and 100 MHz, particularly preferably above 10 MHz and less than 100 MHz. In preferred embodiments, the microwave frequency may be modulated at a rate above 0.5 kHz or 1 kHz, and below 5 kHz or 2 kHz, such as at a frequency of about 1.5 kHz. In preferred embodiments, the microwave frequency may be modulated at an amplitude above 25 MHz or 40 MHZ, and below 85 MHz or 60 MHZ, such as at a frequency of about 50 MHz.

In embodiments of the invention, alpha-KG can advantageously be admixed at appropriate concentration (as described further below) to enable DNP of substantially any 13C-labeled molecules of interest, including for example pyruvic acid, lactic acid, acetic acid, fumaric acid, glutamine, urea, and glucose.

In a preferred embodiment, a glass forming agent such as ethanol, dimethyl sulfoxide, or glycerol may also be added to the solution if required for DNP.

Advantageously, the hyperpolarised solution embodying the invention may be particularly biocompatible because the concentrations of the free-radical precursor and its breakdown products (which are CO₂ gas and succinic acid) after the photo-irradiation and DNP processes are low. The high conversion efficiency on photo-irradiation described above is therefore highly advantageous. Preferably, the alpha-KG concentration in the solution before photo-irradiation may not need to be larger than 500 mM, and is preferably equal to or lower than 400 mM or 300 mM. This is considerably lower than conventional free-radical precursors, and leads to correspondingly low concentrations of the breakdown products.

At the concentrations required for effective hyperpolarisation, both alpha-KG and its breakdown products derived from photo-irradiation can advantageously be safely injected into humans at doses relevant to magnetic resonance technology.

Preferably, the photo-irradiation is performed with a light source that emits radiation in either the ultraviolet spectrum or the visible spectrum or both. The exposure time, which should preferably be less than 5 min, as well as the amount of light power are preferably such as to photo-induce a concentration of radicals between 10 mM and 100 mM, particularly preferably above 30 mM and less than 70 mM. In general the exposure time may be as short as possible in order to improve the rate of processing of the method, but the exposure time may typically be longer than 10 ms or 100 ms.

Preferably, the photo-irradiation should be done at a temperature below 200K, or 190K, particularly preferably above 75K and below 150K, so that the photo-induced free radicals are stable.

After irradiation, the starting solution may be loaded or inserted into a DNP polariser at a high magnetic field (between 3T and 15T, preferably above 5T and below 7T) and a cryogenic environment at a temperature of preferably below 2K. The microwave irradiation may then be started in order to hyperpolarise the frozen solution.

Once the sample has been polarised to an adequate level, the temperature of the frozen solution may be raised to above 200K, preferably between 200K and 273K, within a magnetic field of at least 0.5T, preferably above 1 T and below 7 T, in order to quench the photo-induced radical, namely 2-hydroxyglutaryl radical. Shortly after, preferably less than 1 min after, the temperature of the frozen solution is lowered, preferably to below 78K, particularly preferably to below 40K, and kept in a magnetic field of at least 0.1T, preferably between 0.5 and 5T, in order to minimise any loss of polarisation by spin-lattice relaxation. A low storage temperature and a high magnetic field may advantageously lengthen the storage time while retaining polarisation.

Just prior to an NMR, MRS, or MRI experiment or process in which the hyperpolarised sample is to be used, the frozen solution may be melted or dissolved in a solvent (typically water) or a buffer solution containing a base to adjust the pH of the resulting hyperpolarised solution. Once warmed up to physiologically-compatible temperature, the solution can be directly injected into animals or humans. It should be noted that no filtration process may be required before injection because the free radical has been thermally quenched to form low toxicity endogenous breakdown products, and because the concentration of alpha-KG which was required in the starting solution to generate the free radicals was advantageously low, because of the high efficiency found by the inventors for alpha-KG free-radical generation on photo-irradiation. Therefore the concentration of the breakdown products is correspondingly low.

In summary in a preferred embodiment, the step of irradiating the frozen solution with ultraviolet and/or visible radiation is carried out with the frozen solution at a first temperature below 190K, and the step of hyperpolarising the frozen solution is carried out with the frozen solution at a second temperature below 2K. After hyperpolarisation the temperature of the frozen solution is preferably raised to a third temperature above 200K within a magnetic field of at least 0.5T in order to reduce the concentration of free radicals in the frozen solution, and is then reduced to a fourth temperature below 78 K for storage, advantageously for 15 min or more in a magnetic field of 0.1T or above.

As mentioned above, the storage time for the hyperpolarised sample is very important to the usability of the sample in MR techniques. Currently, in hospitals, the storage time for a hyperpolarised sample may only be one minute, meaning that preparation of samples must be carried out just before use. In embodiments of the invention, the achievable storage time will vary depending on the storage conditions, but the inventors have found that storage times may be as much as an hour, or 10 hours, or up to 48 hours if the storage field is sufficiently large (say 3T) and the temperature is sufficiently low (say below 40K).

As discussed above, pyruvic acid is conventionally used as a molecule of interest for hyperpolarisation, and is itself a free-radical precursor (although it has never been used in humans when polarized using photo-induced free radicals). However, pyruvic acid is a central metabolic substrate and may therefore disadvantageously affect the results of MR scans recorded following its injection. There is therefore a need for a free-radical precursor that can be used in hyperpolarisation of other molecules of interest. The inventors have found that the combination of alpha-KG with frequency modulation of microwaves during polarisation uniquely achieves this, providing advantages including the following:
1. High conversion rate to free radical upon photo-irradiation, which minimizes its concentration in both the starting solution and the final solution;
2. Low toxicity to enable intravenous bolus injections;
3. Well-defined breakdown products with low toxicity, namely CO₂ and succinic acid;
4. Slow cellular uptake to avoid interferences with the biological effect related to the molecule of interest;
5. Yields a photo-induced free radical for which frequency-modulated microwave irradiation leads to unexpectedly high DNP efficiency.

In a further aspect, the invention may advantageously provide a hyperpolarised sample, or solution, for example for use in a magnetic resonance procedure such as NMR, MRS or MRI. The sample can be prepared using a method as described above, and is distinguished from prior-art samples by its low concentration of photo-induced free-radical precursor, namely alpha-KG, and its low concentration of breakdown products from the photo-irradiated precursor, namely succinic acid and carbon dioxide. The sample contains these low concentrations despite the fact that it has not been filtered or otherwise treated to remove any of these materials. This advantageously simplifies the process of using the sample in MR techniques, both reducing the number of failure points in the process and extending the available storage time.

In a preferred embodiment, the invention may thus provide a hyperpolarised solution containing less than 50 mM, preferably less than 25 mM, of alpha-KG, and containing succinic acid at a concentration of less than 20 mM, preferably less than 5 mM.

### DESCRIPTION OF SPECIFIC EMBODIMENTS OF THE INVENTION

Specific embodiments of the invention will now be described by way of example, with reference to the accompanying drawings in which;

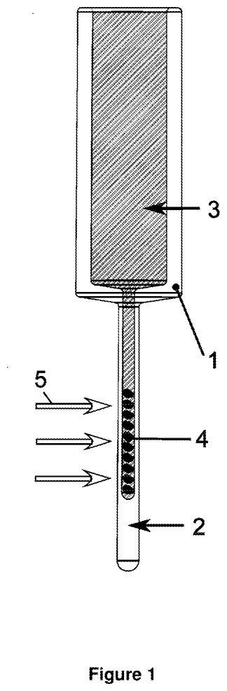

FIG. 1 is a schematic illustration of the formation and UV-Vis irradiation of glassy beads of a sample for low-temperature dynamic nuclear polarization (DNP), according to an embodiment of the invention;

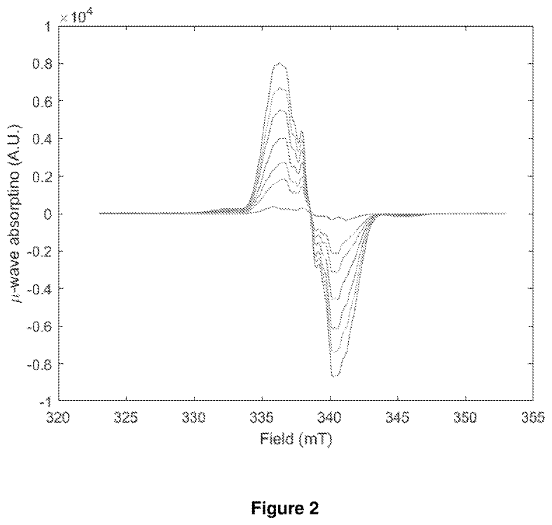

FIG. 2 shows ESR spectra measured at 77 K for different irradiation times of the sample;

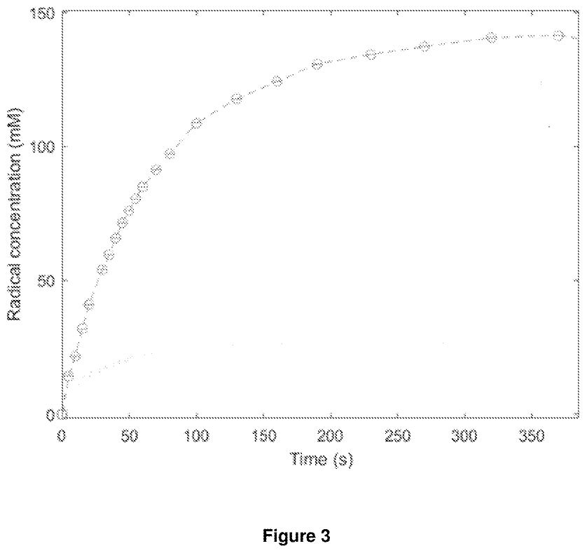

FIG. 3 is a plot of deduced radical concentration as a function of the irradiation time for the sample;

FIG. 4 is a schematic illustration of a DNP polarizer for polarising samples embodying the invention;

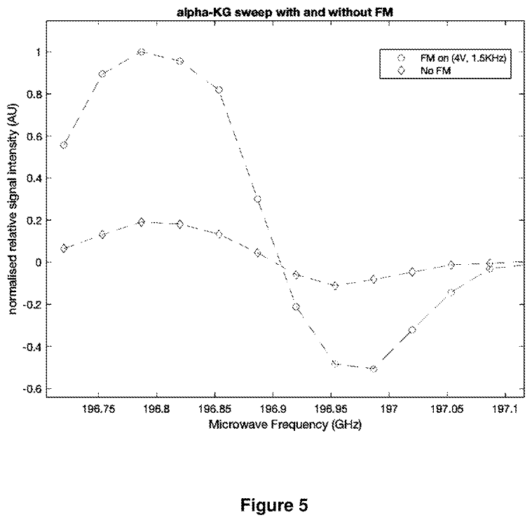

FIG. 5 illustrates a microwave sweep with and without microwave frequency modulation measured inside the DNP polarizer;

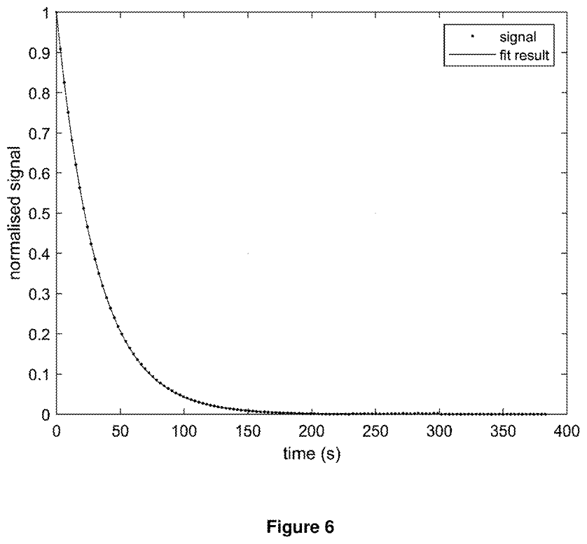

FIG. 6 is a plot of a liquid-state hyperpolarized ¹³C MR signal decay for the sample; and

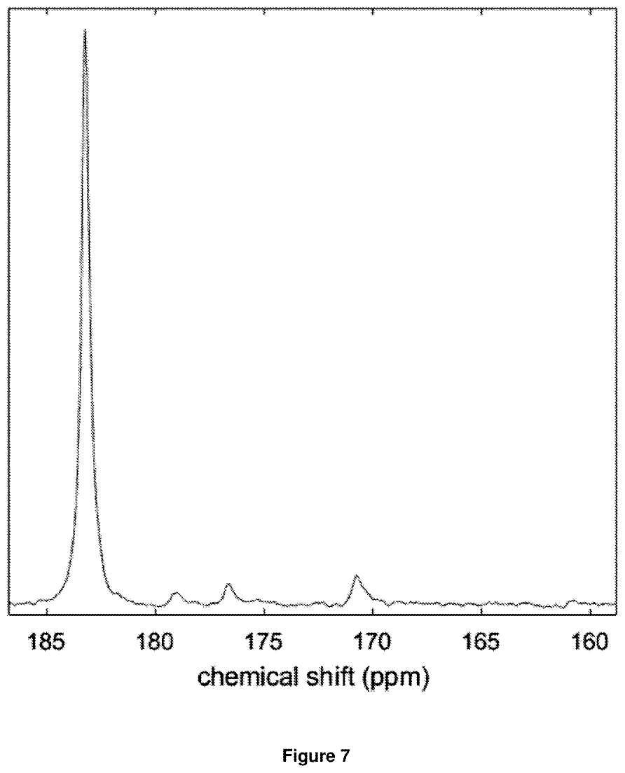

FIG. 7 shows a summed ¹³C MR spectra acquired in a rat liver following the intravenous injection of a hyperpolarised solution embodying the invention.

In a first embodiment of the method of the invention, the first step consists in preparing a starting solution containing alpha-KG and photo-inducing the free radical. FIG. 1 shows a quartz dewar (1) insulated with a vacuum chamber (2) and filled with liquid nitrogen (3). Droplets of a 10M aqueous solution of [1-13C]lactic acid containing 300 mM of alpha-KG are snap frozen in the liquid nitrogen to form glassy beads that fall into the tail of the quartz dewar (4). The glassy frozen beads are subsequently irradiated with UV-Vis light (5) for 30 s using a Dymax Bluewave® 200 W broadband UV-Vis lamp (Dymax, Wiesbaden, Germany) set to maximum output intensity. At the end of the irradiation time, the tail of the quartz dewar can be inserted in an X-band ESR spectrometer to determine the concentration of photo-induced free radicals. FIG. 2 shows the ESR spectra measured at 77 K for various irradiation times for samples irradiated as in FIG. 1 and FIG. 3 is a plot showing the deduced radical concentration as a function of the irradiation time (to deduce the concentration the double integral of the ESR spectrum was compared to a calibration curve, created from a set of known concentrations of the persistent radical compound, 4-hydroxy-2,2,6,6-tetramethylpiperidine-N-oxyl).

The second step of the method consists in polarizing the ¹³C spins of the sample by low-temperature dynamic nuclear polarization (DNP) using a polarizer such as the one sketched in FIG. 4. The polarizer comprises a liquid helium cryostat (6), a superconducting magnet (7), and a frequency-modulated microwave source (8) connected to a waveguide (9) directing the microwaves inside the sample space (10) of the polarizer. The sample space is filled with liquid helium. In a preferred embodiment, the sample, in the form of frozen beads, (11) is enclosed in a leak-tight assembly (12) through which a fluid such as a liquid, such as hot water, or a gas, such as helium or nitrogen gas, can be supplied from an entry port (13) to an exit port (14) to raise the temperature of the sample above 200 K once the sample is sufficiently polarized and the assembly containing the sample is raised out of the liquid helium bath of the sample space. The photo-induced radicals will then be quenched and the sample can either be extracted from the polarizer and directly used for MR experiments, or be extracted from the polarizer for storage in an external device and subsequently used for MR experiments, or be placed back in the sample space of the polarizer for storage.

FIG. 5 displays microwave sweeps with and without microwave frequency modulation measured inside the DNP polarizer. Comparing these measurements clearly illustrates the unexpectedly large beneficial effect of frequency modulation in embodiments of the invention. The sweeps are shown in FIG. 5 as plots of 13C solid-state NMR signal vs. center microwave frequency measured in a photo-irradiated frozen [1-13C]lactic acid solution containing 300 mM of alpha-KG polarized via DNP in a 7 T and 1.35 K polarizer. The microwave was frequency modulated at a rate of 1.5 kHz and an amplitude of 52 MHz. The dashed line linking the measurement points is to guide the eye.

FIG. 6 is an example of liquid-state hyperpolarized ¹³C MR signal decay measured at 14.1 T (a 10-degree radiofrequency pulse was applied every 3 s) in a room-temperature [1-13C]lactate solution hyperpolarized using the free-radical photo-induced in alpha-KG.

FIG. 7 shows the sum of a series of ¹³C MR spectra acquired in a rat liver following the intravenous injection of a sample embodying the invention. The sample was 1 ml of a 42 mM [1-13C]lactate solution hyperpolarized using the free-radical photo-induced in alpha-KG. In addition to [1-13C]lactate (182.8 ppm), the following downstream metabolites could be detected: [1-13C]pyruvate (170.7 ppm) and [1-13C]pyruvate hydrate (179 ppm), [1-13C]alanine (176.7 ppm), [1-13C]malate (175.2 ppm), and [13C]bicarbonate (160.8 ppm). The data was acquired in preclinical horizontal-bore 9.4T MRI system using a 10-mm diameter 13C surface coil and a series of 20-degree radiofrequency pulses applied every 2 s.

1. A method for preparing a hyperpolarised sample comprising the steps of:
freezing a solution comprising alpha-ketoglutaric acid and ¹³C-labelled molecules to form a frozen solution;
irradiating the frozen solution with ultraviolet and/or visible radiation;
hyperpolarising the frozen solution by applying a magnetic field to the solution while irradiating the frozen solution with frequency-modulated microwave radiation.

2. A method according to claim 1, in which the microwave radiation is frequency-modulated at a rate between 1 Hz and 1 MHz with an amplitude between 1 Hz and 100 MHz.

3. A method according to claim 1, in which the concentration of alpha-ketoglutaric acid in the frozen solution is in the range of 10 mM to 500 mM.

4. A method according to claim 1, in which the step of irradiating with ultraviolet and/or visible radiation generates free radicals in the frozen solution.

5. A method according to claim 4, in which a free-radical concentration of 10-100 mM is generated in the frozen solution.

6. A method according to claim 1, in which the step of freezing the solution comprises reducing the temperature of the solution to below 190K.

7. A method according to claim 1, in which the magnetic field strength is between 3 and 15 T.

8. A method according to claim 1, in which the ¹³C-labelled molecules comprise one or more of pyruvic acid, lactic acid, acetic acid, glutamine, fumaric acid, urea, or glucose.

9. A method according to claim 1, in which the step of irradiating the frozen solution with ultraviolet and/or visible radiation is carried out with the frozen solution at a first temperature below 190K, and the step of hyperpolarising the frozen solution is carried out with the frozen solution at a second temperature below 2K.

10. A method according to claim 9, in which after hyperpolarisation the temperature of the frozen solution is raised to a third temperature above 200K within a magnetic field of at least 0.5T in order to reduce the concentration of free radicals in the frozen solution, and is then reduced to a fourth temperature below 78 K for storage in a magnetic field of 0.1T or above.

11. A method according to claim 1, in which the solution further comprises a glass forming agent, preferably ethanol, dimethyl sulfoxide or glycerol.

12. A method according to claim 1, further comprising the step of dissolving or melting the polarised frozen solution for use in a magnetic resonance application such as NMR, MRS or MRI.

13. A hyperpolarised sample for use in NMR, MRS or MRI prepared according to a method as defined in claim 1.

14. A hyperpolarised sample for use in NMR, MRS or MRI containing alpha-KG at a concentration of less than 50 mM and containing succinic acid at a concentration of less than 20 mM.

15. A hyperpolarised sample according to claim 14 containing alpha-KG at a concentration of less than 25 mM and/or containing succinic acid at a concentration of less than 5 mM.
